(19) United States
(12) Patent Application Publication
(10) Pub. No.: US 2024/0382134 A1
(43) Pub. Date: Nov. 21, 2024

(54) BRAIN ACTIVITY ANALYSIS SYSTEM, BRAIN ACTIVITY ANALYSIS METHOD, AND COMPUTER-READABLE STORAGE MEDIUM

(71) Applicant: JVCKENWOOD Corporation, Yokohama-shi (JP)

(72) Inventor: Hayato Nakao, Yokohama-shi (JP)

(21) Appl. No.: 18/786,643

(22) Filed: Jul. 29, 2024

Related U.S. Application Data
(63) Continuation of application No. PCT/JP2022/041165, filed on Nov. 4, 2022.

(30) Foreign Application Priority Data
Feb. 24, 2022 (JP) 2022-026967

(51) Int. Cl. A61B 5/291; A61B 5/00; A61B 5/05; A61B 5/0537

(52) U.S. Cl. CPC A61B 5/291; A61B 5/05; A61B 5/0537; A61B 5/4064

(57) ABSTRACT

A brain activity analysis system includes: a first measurement unit configured to measure an intracerebral signal based on a state of a brain; a second measurement unit configured to measure a biological reaction signal generated in a body site other than the brain in association with brain activity; a first analysis unit configured to estimate a first range indicating a range of a source of the intracerebral signal based on measurement data by the first measurement unit; a second analysis unit configured to estimate a second range indicating the range of the source of the intracerebral signal based on the measurement data by the second measurement unit; and a processing unit configured to calculate an estimated position of the source of the intracerebral signal based on the first range and the second range, and output information regarding the calculated estimated position.

### CROSS-REFERENCE TO RELATED APPLICATION

This application is a Continuation of PCT International Application No. PCT/JP2022/041165 filed on Nov. 4, 2022 which claims the benefit of priority from Japanese Patent Application No. 2022-026967 filed on Feb. 24, 2022, the entire contents of both of which are incorporated herein by reference.

### BACKGROUND

The present disclosure relates to a brain activity analysis system, a brain activity analysis method, and a computer-readable storage medium.

In recent years, the application of brain science to medical care and healthcare has progressed, and there is an increasing need for analysis and visualization of brain information. As a method for analyzing a signal source of a brain activity using an electroencephalograph or a magnetoencephalograph, an equivalent current dipole method or a spatial filter method is known. For example, Japanese Patent No. 5077823 discloses a brain activity analysis method for estimating a signal source in a brain by a spatial filter method and displaying the estimated signal source on a display device. In the brain activity analysis method, when a signal from a signal source is estimated and displayed, only a portion where a maximum value and a maximal value are spatially extracted and averaged and displayed.

Meanwhile, since spatial resolution of signal source estimation depends on a signal-to-noise ratio (S/N), when an electroencephalograph or a magnetoencephalograph is used in a noisy environment, there is a problem that the spatial resolution of the signal source estimation decreases.

### SUMMARY

A brain activity analysis system according to one aspect of the present disclosure includes: a first measurement unit configured to measure an intracerebral signal based on a state of a brain; a second measurement unit configured to measure a biological reaction signal generated in a body site other than the brain in association with brain activity; a first analysis unit configured to estimate a first range indicating a range of a source of the intracerebral signal based on measurement data by the first measurement unit; a second analysis unit configured to estimate a second range indicating the range of the source of the intracerebral signal based on the measurement data by the second measurement unit; and a processing unit configured to calculate an estimated position of the source of the intracerebral signal based on the first range and the second range, and output information regarding the calculated estimated position.

A brain activity analysis method according to another aspect of the present disclosure includes: measuring an intracerebral signal based on a state of a brain; estimating a first range indicating a range of a source of the intracerebral signal based on measurement data of the intracerebral signal; measuring a biological reaction signal generated in a body site other than the brain in association with brain activity; estimating a second range indicating the range of the source of the intracerebral signal based on measurement data of the biological reaction signal generated in association with the brain activity; calculating an estimated position of the source of the intracerebral signal based on the first range and the second range; and outputting information regarding the calculated estimated position.

A non-transitory computer-readable storage medium according to still another aspect of the present disclosure stores a program causing a computer to execute: measuring an intracerebral signal based on a state of a brain; estimating a first range indicating a range of a source of the intracerebral signal based on measurement data of the intracerebral signal; measuring a biological reaction signal generated in a body site other than the brain in association with brain activity; estimating a second range indicating the range of the source of the intracerebral signal based on measurement data of the biological reaction signal generated in association with the brain activity; calculating an estimated position of the source of the intracerebral signal based on the first range and the second range; and outputting information regarding the calculated estimated position.

### BRIEF DESCRIPTION OF THE DRAWINGS

FIG. 1 is a diagram schematically illustrating a configuration example of a brain activity analysis system according to the present disclosure;

FIG. 2 is a diagram illustrating a relationship between a biological reaction generated in association with a brain activity, an occurrence place of the brain activity, and a biological reaction measurement unit;

FIG. 3 is a flowchart illustrating a flow of a brain activity analysis method according to the first embodiment;

FIG. 4 is a diagram illustrating a first display mode of the brain activity analysis system according to the first embodiment;

FIG. 5 is a diagram illustrating a second display mode of the brain activity analysis system according to the first embodiment;

FIG. 6 is a diagram illustrating a third display mode of the brain activity analysis system according to the first embodiment;

FIG. 7 is a flowchart illustrating a flow of a brain activity analysis method according to a second embodiment;

FIG. 8 is a diagram illustrating a first display mode of a brain activity analysis system according to a third embodiment; and

FIG. 9 is a diagram illustrating a second display mode of the brain activity analysis system according to the third embodiment.

### DETAILED DESCRIPTION

Hereinafter, embodiments of the present disclosure will be described in detail with reference to the drawings. Note that the present disclosure is not limited to the embodiments described below.

### First Embodiment

### Configuration of Brain Activity Analysis System

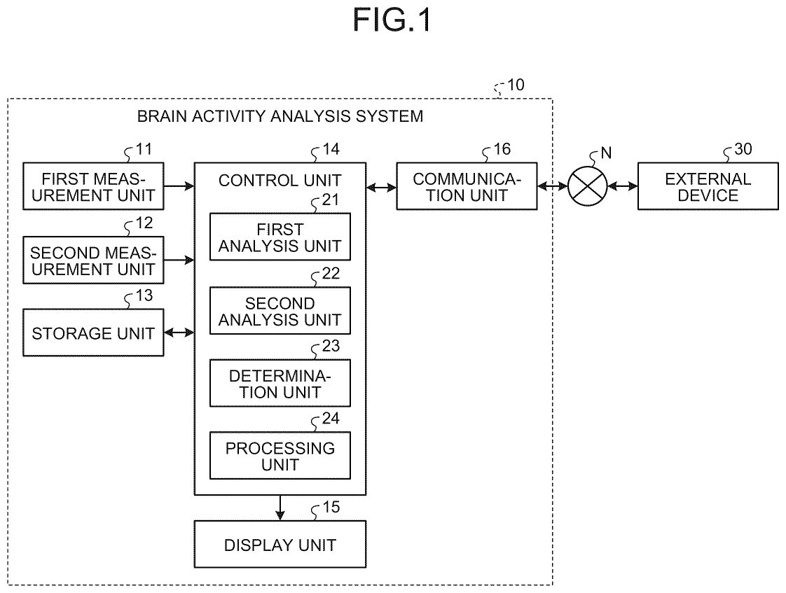

FIG. 1 is a diagram schematically illustrating a configuration example of a brain activity analysis system according to the present disclosure.

As illustrated in FIG. 1, a brain activity analysis system 10 according to the present disclosure includes a first measurement unit 11, a second measurement unit 12, a storage unit 13, a control unit 14, a display unit 15, and a communication unit 16. Note that the brain activity analysis system 10 is communicably connected to an external device 30 via a network N.

The first measurement unit 11 is a measurement instrument that measures an intracerebral signal based on the state of the brain. The first measurement unit 11 may be, for example, an electroencephalography (EEG) or a magnetoencephalography (MEG). The electroencephalograph applies an electrode that derives a biological action potential (around 20 μV) generated by activity of nerve cells in the brain to a scalp, amplifies a signal received by the electrode, and measures the biological action potential of the nerve cells in the brain. For example, the electroencephalograph may be realized by a type in which a contact resistance between the electrode and the scalp is reduced by disposing a conductive gel between the scalp and the electrode, a type in which a sponge is provided between the scalp and the electrode to contain an electrolytic solution in the sponge, a type in which an amplifier is built in the vicinity of the electrode to receive an electric signal with high input impedance and output the electric signal with low output impedance, or the like.

In the magnetoencephalograph, a large number of measurement coils are arranged on a scalp, and an induced current flowing through the measurement coils is measured by a magnetic field generated by biological activity of nerve cells. As the magnetoencephalograph, a tunnel magneto resistance (TMR) element may be used instead of the measurement coil. The tunnel magnetoresistive element includes a tunnel junction in which an insulator is sandwiched between two ferromagnets. A magnetic field is measured using a property in which an occurrence probability of tunnel conduction passing through an insulator of electrons is different depending on a spin state (up-spin or down-spin) of electrons. In addition, a superconductive quantum interface device (SQUID) may be used instead of the measurement coil of the magnetoencephalograph. The superconductive quantum interface device is a highly sensitive magnetic sensor capable of detecting an extremely weak magnetic field by using an element including a Josephson junction in an annular superconductor. The Josephson junction is a junction having a structure in which a thin normal conductor or an insulator is sandwiched between superconductors. In the superconducting state, when a magnetic field is applied to the annular superconductor from the outside, an interruption current that cancels the magnetic field flows so as not to pass the magnetic field into the annular superconductor. When the annular superconductor has the Josephson junction, a superconducting state collapses in the Josephson junction only by a slight interruption current flowing, electric resistance is generated, and a voltage is generated. By measuring this voltage, a slight change in the magnetic field can be measured.

The second measurement unit 12 measures a reaction of nerve cells in the brain, that is, a biological reaction signal generated in a body site other than the brain in association with brain activity.

The second measurement unit 12 may be, for example, an electro dermal activity (EDA) sensor that detects an electric actibity state of a skin. The electro dermal activity sensor measures an electrical activity state of the skin due to sweat secreted from sweat glands (for example, eccrine glands) of the skin. Electro dermal activity is roughly divided into electrodermal potential and skin conductance. The electrodermal potential is distinguished into an electrodermal level and electrodermal reflex. The electrodermal level is a direct-current component of the electrodermal potential, and when a wakefulness level is high, the electrodermal activity level illustrates a negative high value. A positive value is illustrated in a case where a user feels drowsy or in a relaxed state. The electrodermal reflex is an alternating component of the electrodermal potential, and the electrodermal reflex frequently occurs when a stimulus such as pain sensation, tactile sensation, auditory sensation, or visual sensation occurs, or when deep breathing, body movement, mental calculation, or thinking is performed.

The second measurement unit 12 may be, for example, a pulse wave sensor that detects pulse wave information. The pulse wave sensor is a sensor that captures, as a waveform, a change in volume of a blood vessel that occurs as the heart pumps blood. The pulse wave sensor irradiates a living body with infrared light, red light, or light having a green wavelength around 550 nm, and measures light transmitted or reflected in the living body using a photodiode or a phototransistor. Since oxygenated hemoglobin exists in blood of an artery and has a property of absorbing incident light, it is possible to obtain a pulse wave signal by measuring a blood volume (volume change of blood vessel) that changes with pulsation of the heart in time series.

The second measurement unit 12 may be, for example, a camera that detects pupil reflection. The camera includes an optical element and an imaging element. The optical element is an element constituting an optical system such as a lens, a mirror, a prism, or a filter. The imaging element is an element that converts light incident through the optical element into an image signal that is an electric signal. The imaging element is, for example, a charge coupled device (CCD) sensor, a complementary metal oxide semiconductor (CMOS) sensor, or the like.

The storage unit 13 includes a main storage device and an external storage device. The main storage device stores a program executed by the control unit 14 or data processed by the control unit 14. The main storage device may be realized by, for example, a semiconductor memory element such as a random access memory (RAM), a read only memory (ROM), a flash memory, or the like. The external storage device stores data to be processed by the control unit 14. The external storage device may be realized by, for example, a hard disk, a solid state drive (SSD), an optical disk, or the like.

The control unit 14 is implemented by a central processing unit (CPU), a micro processing unit (MPU), or the like executing various programs stored in the storage unit 13 using a RAM as a work area. Furthermore, the control unit 14 may be realized by, for example, an integrated circuit such as an application specific integrated circuit (ASIC) or a field programmable gate array (FPGA), an analog circuit using an electronic circuit such as an operational amplifier, or the like.

The control unit 14 includes a first analysis unit 21, a second analysis unit 22, a determination unit 23, and a processing unit 24. The control unit 14 can also be called a brain activity analysis device that executes processing of the first analysis unit 21, the second analysis unit 22, the determination unit 23, and the processing unit 24. For example, the control unit 14 reads and executes a program (software) from the storage unit 13 to implement the first analysis unit 21, the second analysis unit 22, the determination unit 23, and the processing unit 24, and executes these processes. Note that the control unit 14 may execute these processes by one CPU, or may include a plurality of CPUs and execute the processes by the plurality of CPUs. In addition, at least some of these processes may be realized by an integrated circuit, an analog circuit, or the like.

The first analysis unit 21 estimates a first range indicating a range of a source of the intracerebral signal based on measurement data by the first measurement unit 11. For example, in a case where the first measurement unit 11 is an electroencephalograph, the first analysis unit 21 estimates the source based on the position of the electrode from which the electroencephalogram has been detected. The electroencephalograph measures biological action potentials of nerve cells in the brain by placing 21 electrodes on the scalp. The arrangement of the electrodes is determined by a method called International 10-20 method, and the potential on the scalp is measured using 2 earlobes as a reference potential. Therefore, the source of the electroencephalogram can be estimated by the position of the electrode where the potential change is detected. However, the arrangement of the electrodes may be arbitrary.

When the first measurement unit 11 is an electroencephalograph and a magnetoencephalograph, the first analysis unit 21 estimates a first range indicating a range of a source of an intracerebral signal by a spatial filter method. In the spatial filtering method, a region of interest in the skull is divided into a lattice shape, and a signal outside the region of interest is removed as noise. This makes it possible to detect a weak signal buried in the interference signal. In addition, in a case where the first measurement unit 11 is an electroencephalograph and a magnetoencephalograph, the first analysis unit 21 may use an equivalent current dipole (ECD) method for obtaining a current dipole that most closely approximates each of a potential and a magnetic field measured on the scalp. In the equivalent current dipole method, a current dipole that minimizes the root mean square of the difference between the potential at which the dipole is generated at the electrode position and the measured potential is obtained.

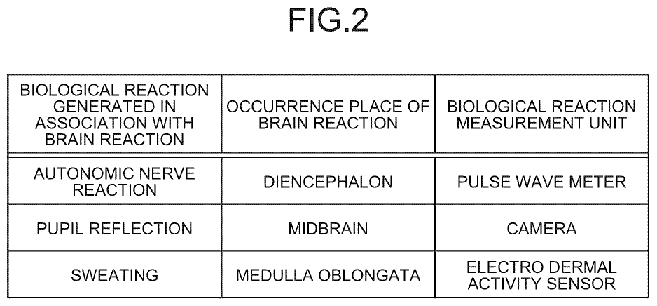

The second analysis unit 22 estimates a second range indicating the range of the source of the intracerebral signal based on the measurement data by the second measurement unit 12. A method of estimating the source of the intracerebral signal by the second analysis unit 22 will be described with reference to FIG. 2. FIG. 2 is a diagram illustrating a relationship between a biological reaction generated in association with the brain activity, an occurrence place of the brain activity, and a biological reaction measurement unit. For example, the second analysis unit 22 may read and acquire the correspondence relationship between the type of the biological reaction signal and the generation source of the intracerebral signal from the storage unit 13 or the like, and estimate the source of the intracerebral signal based on the type of the biological reaction signal measured by the second measurement unit 12 and the correspondence relationship. For example, as illustrated in FIG. 2, in a case where the biological reaction signal corresponding to the autonomic nerve reaction is detected by the pulse wave meter, the second analysis unit 22 estimates the source of the intracerebral signal as “diencephalon”. Furthermore, in a case where the pupil reflection is detected by the camera, the second analysis unit 22 estimates the source of the intracerebral signal as “midbrain”. When the biological reaction signal corresponding to sweating is detected by the electro dermal activity sensor, the second analysis unit 22 estimates the source of the intracerebral signal as “medulla oblongata”. However, the correspondence relationship between the biological reaction signal and the occurrence place of the brain activity illustrated in FIG. 2 is an example, and the estimation may be performed based on any other correspondence relationship.

The second measurement unit 12 measures a biological reaction signal generated in association with the brain activity. Therefore, for example, in a case where the biological reaction signal is detected by the second measurement unit 12 with a delay of a predetermined time from the time point at which the electroencephalogram is detected by the first measurement unit 11, the source of the intracerebral signal corresponding to the biological reaction signal detected by the second measurement unit 12 may be estimated as the source of the intracerebral signal estimated by the second analysis unit 22. Note that the predetermined time in this case may be arbitrarily set according to the type of the biological reaction signal. For example, in the case of the pulse wave signal, the pulse wave signal in association with the intracerebral signal may be detected with a delay of about 3 seconds from the detection of the intracerebral signal.

It can also be said that the second analysis unit 22 associates the intracerebral signal measured by the first measurement unit 11 with the biological reaction signal measured by the second measurement unit 12. More specifically, the second analysis unit 22 determines which intracerebral signal is accompanied by the biological reaction signal measured by the second measurement unit 12, and estimates the second range based on the determination result. The second analysis unit 22 may associate the intracerebral signal with the biological reaction signal by an arbitrary method, but for example, may determine that the biological reaction signal detected within a predetermined time from the time when the intracerebral signal is detected is accompanied with the intracerebral signal. Furthermore, for example, the period from the time at which the intracerebral signal occurs to the time at which the biological reaction occurs may vary depending on the type of biological reaction. Therefore, the second analysis unit 22 may set a predetermined time based on the type of the biological reaction signal, and associate the intracerebral signal detected at a time a predetermined time before the time at which the biological reaction signal is detected with the biological reaction signal. A method of setting the predetermined time based on the type of the biological reaction signal is arbitrary, but for example, a correspondence relationship between the type of the biological reaction signal and the predetermined time may be set in advance.

The determination unit 23 determines whether the first range indicating the range of the source of the intracerebral signal estimated based on the measurement data by the first measurement unit 11 overlaps the second range indicating the range of the source of the intracerebral signal based on the measurement data by the second measurement unit 12. Here, the overlap between the first range and the second range refers to a state in which a part or the whole of the second range is included in the first range or a state in which a part or the whole of the first range is included in the second range. The determination unit 23 preferably determines whether the first range and the second range overlap each other for the intracerebral signal and the biological reaction signal associated by the second analysis unit 22. In the first range estimated by the first analysis unit 21, the source of the intracerebral signal is estimated with a predetermined spread. Meanwhile, as illustrated in FIG. 2, the second range indicates predetermined portions in the brain such as “diencephalon”, “midbrain”, and “medulla oblongata”.

The processing unit 24 calculates the estimated position of the source of the intracerebral signal based on the first range and the second range, and outputs information regarding the calculated estimated position. Note that the information regarding the estimated position output by the processing unit 24 will be described later, but is not limited to the information described later and may be arbitrary. For example, information to which evaluation of brain activity that can be used by a doctor for diagnosis of brain activity is added may be output.

The display unit 15 displays the source of the intracerebral signal based on the estimated position estimated by the processing unit 24. The display unit 15 may be a Cathode-Ray Tube (CRT) display, a liquid crystal display, a plasma display, an organic Electro Luminescence (EL) display, a micro Light Emitting Diode (LED) display, or the like.

The communication unit 16 communicably connects the inside and the outside of the brain activity analysis system 10, and transmits and receives information between the inside and the outside. The communication unit 16 is realized by, for example, a network interface card (NIC), a wireless local area network (LAN) card, or the like. Then, the communication unit 16 is connected to a network N to be described later in a wired or wireless manner, and transmits and receives information to and from an external device 30 to be described later.

The network N is a communication network that transmits and receives information in a wired or wireless manner. In the wired case, Ethernet (registered trademark) (ETHERNET (registered trademark)) specified in IEEE802.3 may be used. The wireless communication may be realized by using a wireless LAN, Bluetooth (registered trademark), a 4th generation mobile communication system (4G), or a 5th generation mobile communication system (5G) specified in IEEE802.11.

The external device 30 is an information processing device communicably connected to the brain activity analysis system 10 via the network N. The external device 30 may be, for example, a smartphone, a tablet terminal, a desktop personal computer (PC), a notebook PC, a mobile phone, a personal digital assistant (PDA), or the like.

The external device 30 receives data processed by the processing unit 24 via the communication unit 16 and the network N of the brain activity analysis system 10. Then, the data processed by the processing unit 24 may be displayed by a display device included in the external device 30, or may be used for various applications such as a sleep application by the external device 30, for example.

### Processing of Outputting Information Regarding Estimated Position

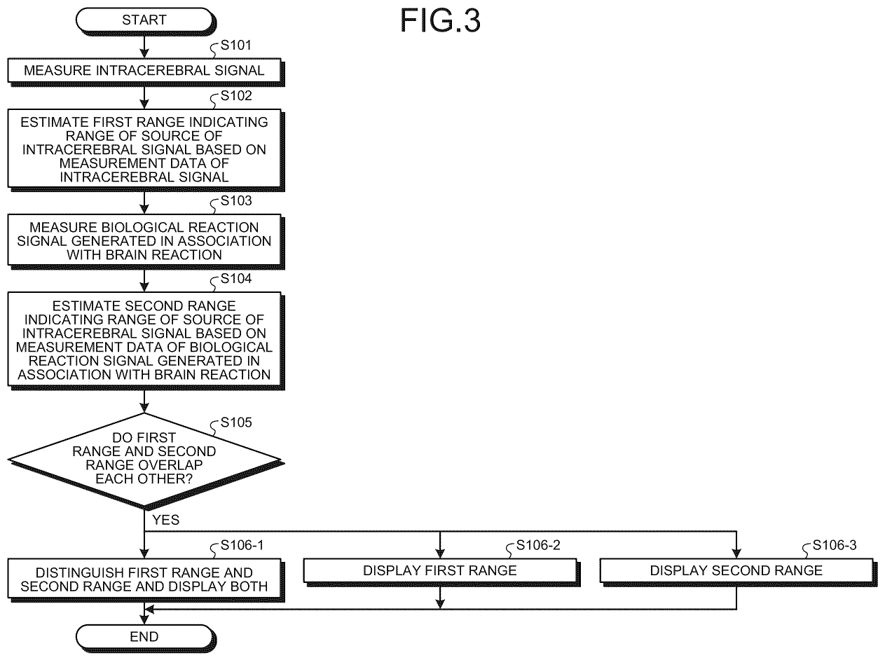

Next, a brain activity analysis method (processing of outputting information regarding an estimated position) according to the present embodiment will be described with reference to FIG. 3. FIG. 3 is a flowchart illustrating a flow of a brain activity analysis method according to the first embodiment. With reference to FIG. 3, the brain activity analysis method according to the present disclosure will be described step by step.

First, in the brain activity analysis method according to the first embodiment, the first measurement unit 11 measures the intracerebral signal (Step S101). Next, the first analysis unit 21 acquires the intracerebral signal measured by the first measurement unit 11, and estimates the first range indicating the range of the source of the intracerebral signal based on the acquired measurement data of the intracerebral signal (Step S102). Then, the second measurement unit 12 measures the biological reaction signal generated in association with the brain activity (Step S103). Then, the second analysis unit 22 acquires the biological reaction signal measured by the second measurement unit 12, and estimates the second range indicating the range of the source of the intracerebral signal based on the measurement data of the biological reaction signal generated in association with the brain activity (Step S104). Then, the processing unit 24 determines whether the first range and the second range overlap each other (Step S105). When the first range and the second range overlap each other (Step S105; YES), the first range and the second range are distinguished and displayed (Step S106-1), the first range is displayed (Step S106-2), or the second range is displayed (Step S106-3). That is, in Step S106, which of the first range and the second range is to be displayed can be set in advance, or can be manually switched by the user each time.

The case of selecting to display both the first range and the second range (Step S106-1) is, for example, a case of notifying the user that the first range is an accurate signal source. The case of selecting to display the first range (Step S106-2) is, for example, a case where the first range is included in the second range. For example, when the occurrence places of the brain activity determined by the biological reaction signal signals vary in size, for example, when the cerebellum is larger than other portions and the second range is estimated to be the cerebellum, the first range may be smaller than the cerebellum. By displaying the first range, the purpose of improving the estimation accuracy of the signal source can be achieved. In addition, since the first range and the second range overlap each other, the first range and the second range coincide with each other, and it is considered that the first range estimated based on the measurement data of the first measurement unit 11 is appropriate. That is, it can be confirmed that the influence of noise or the like on the first measurement unit 11 is small. In a case where the display of the second range (Step S106-3) is selected, for example, the second range is narrower, so that the purpose of improving the estimation accuracy of the signal source can be achieved. When the first range and the second range do not overlap each other (Step S105; NO), as in the second embodiment described later, the first range and the second range may be distinguished and displayed (Step S106-1).

Here, an example in which the display unit 15 distinguishes and displays the first range and the second range will be described. That is, the display unit 15 displays the first range and the second range in different modes. In other words, the display unit 15 displays the first range and the second range in different display modes.

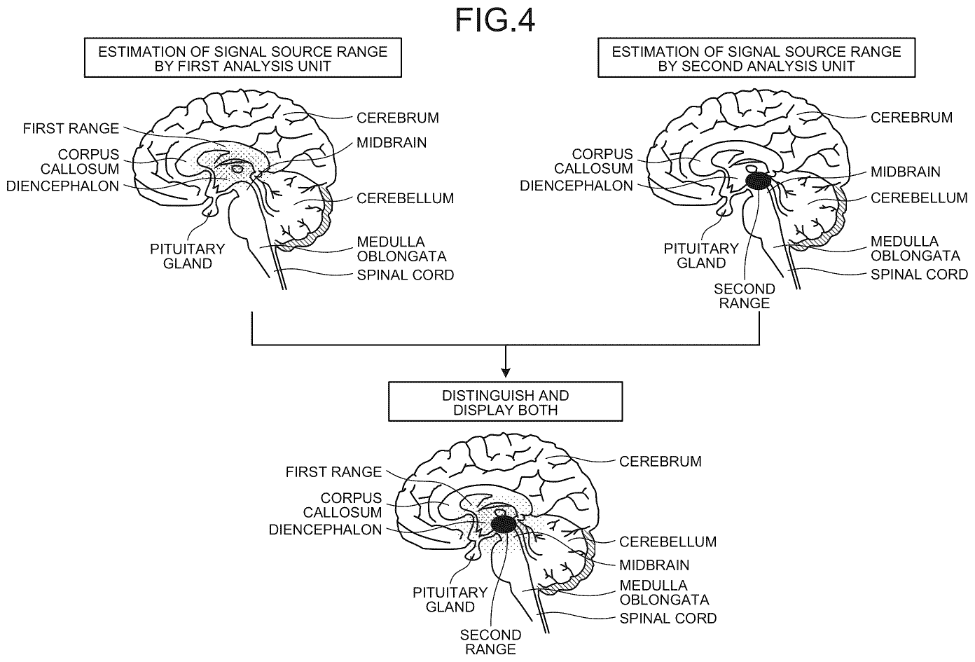

FIG. 4 is a diagram illustrating a first display mode of the brain activity analysis system according to the first embodiment. As illustrated in FIG. 4, the brain activity analysis system 10 according to the first embodiment distinguishes and displays the first range estimated by the first analysis unit 21 and the second range estimated by the second analysis unit 22. For example, the first range and the second range may be displayed in different colors, or may be displayed using different hatching.

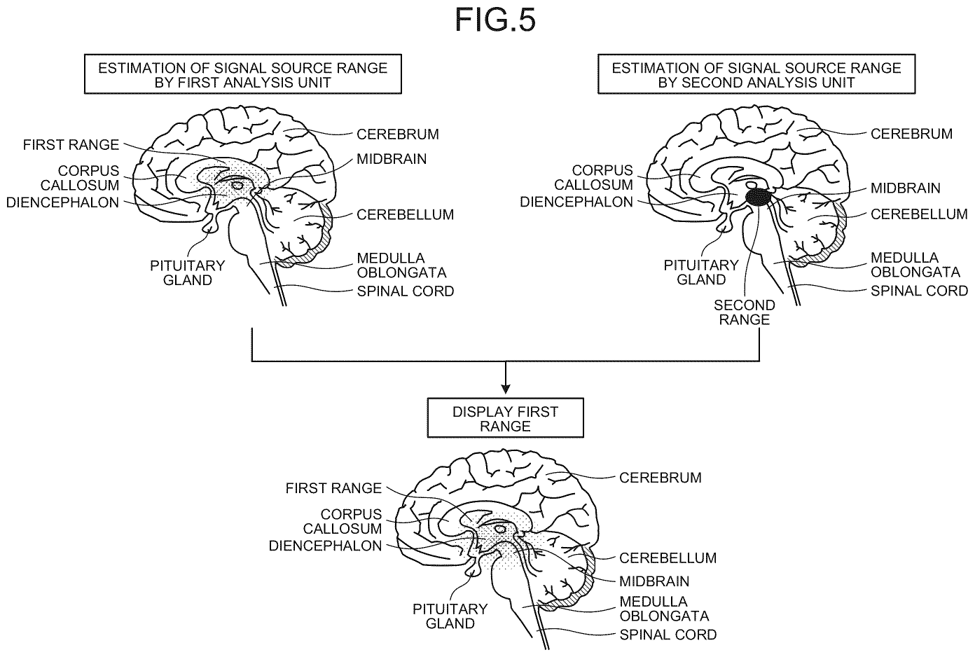

FIG. 5 is a diagram illustrating a second display mode of the brain activity analysis system according to the first embodiment. As illustrated in FIG. 5, in the second display mode, the brain activity analysis system 10 displays the first range. As a result, the purpose of improving the estimation accuracy of the signal source based on the first measurement unit 11 can be achieved.

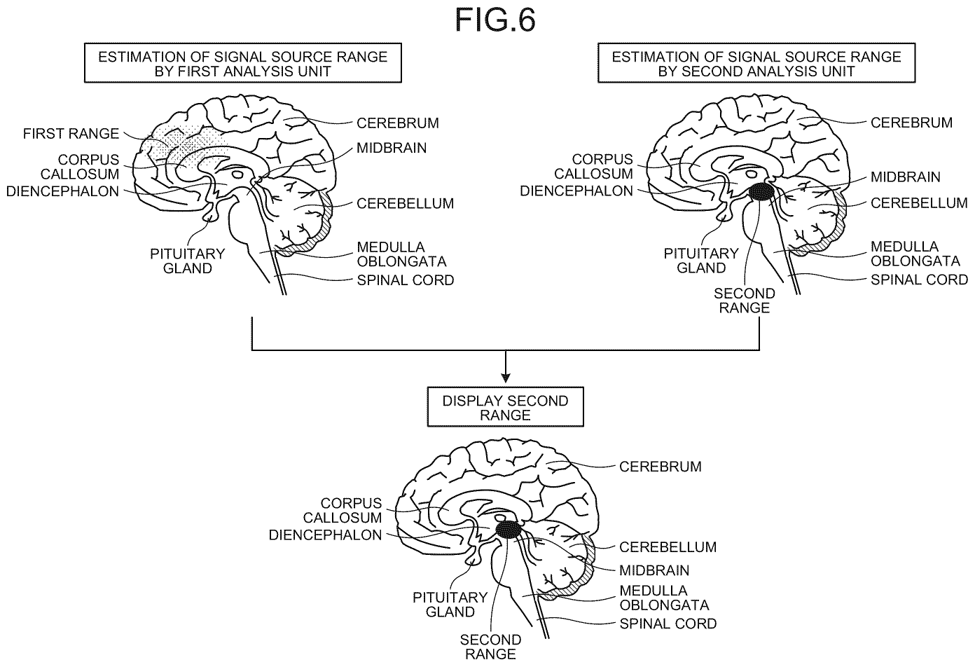

FIG. 6 is a diagram illustrating a third display mode of the brain activity analysis system according to the first embodiment. As illustrated in FIG. 6, in the third display mode, the brain activity analysis system 10 displays the second range. As described above, this display mode is, for example, a case where the second range is narrower, and thus, the purpose of improving the estimation accuracy of the signal source can be achieved.

Note that the above-described brain activity analysis method may be executed using the brain activity analysis system 10, or may be executed by any other means.

Furthermore, the above-described brain activity analysis method can also be executed by a program executed by a computer included in the control unit 14.

As a result, the second range indicating the range of the source of the intracerebral signal is estimated based on the biological reaction signal, and both the first range indicating the source of the intracerebral signal estimated based on the intracerebral signal and the second range are used for estimation of the source of the intracerebral signal, so that the estimation accuracy of the source of the intracerebral signal can be improved.

### Second Embodiment

Next, a brain activity analysis system 10 according to a second embodiment will be described. The brain activity analysis system 10 according to the second embodiment is different from the brain activity analysis system 10 according to the first embodiment in the processing of the processing unit 24. Therefore, in the configuration according to the second embodiment, points different from the configuration according to the first embodiment will be described.

When the first range includes the second range, the processing unit 24 sets the second range as the estimated position. The processing unit 24 according to the first embodiment sets the first range as the estimated position when the second range is included in the first range, but the processing unit 24 according to the second embodiment sets the second range as the estimated position when the second range is included in the first range as described above.

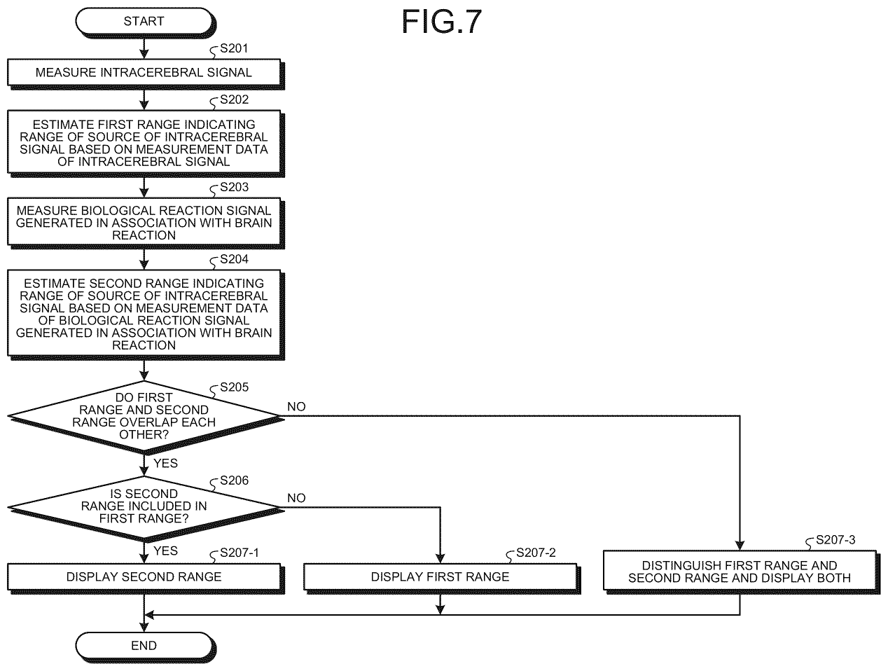

Next, the brain activity analysis method according to the second embodiment will be described with reference to FIG. 7. FIG. 7 is a flowchart illustrating a flow of the brain activity analysis method according to the second embodiment. With reference to FIG. 7, a brain activity analysis method according to the second embodiment will be described step by step. In the brain activity analysis method according to the second embodiment, Steps S201 to S204 are the same as Steps S101 to S104 of the brain activity analysis method according to the first embodiment, and thus the description of Steps S201 to S204 is omitted.

In the brain activity analysis method according to the second embodiment, after Steps S201 to S204 that are the same as Steps S101 to S104 of the brain activity analysis method according to the second embodiment are executed, it is determined whether the first range and the second range overlap each other (Step S205). When the first range and the second range overlap (Step S205; YES), the determination unit 23 determines whether the second range is included in the first range (Step S206). When the second range is included in the first range (Step S206; YES), the second range is displayed as information indicating the estimated position (Step S207-1). When the second range is not included in the first range (Step S206; NO), the first range is displayed as information indicating the estimated position (Step S207-2).

When the first range and the second range do not overlap each other (Step S205; NO), the first range and the second range are distinguished and displayed (Step S207-3). Thus, by displaying both, it is possible to notify the user of the possibility that the first range is not an accurate signal source. Note that, in Step S205, as the cause of the mismatch between the first range and the second range, erroneous estimation of the first range due to noise mixing into the first measurement unit 11 due to use in an environment with a lot of noise, and erroneous estimation of the first range due to noise mixing into the first measurement unit 11 due to defective attachment of an electrode or a magnetic sensor are assumed.

The display mode of the brain activity analysis system 10 according to the second embodiment is displayed in three display modes illustrated in FIGS. 4, 5, and 6, similarly to the display mode of the brain activity analysis system 10 according to the first embodiment. Therefore, the description of the display mode of the brain activity analysis system 10 according to the second embodiment will be omitted.

As a result, since the first range and the second range can be compared with each other, it is possible to confirm whether the first range exists and to further improve the accuracy of the first range.

### Third Embodiment

Next, a brain activity analysis system 10 according to a third embodiment will be described. The brain activity analysis system 10 according to the third embodiment is different from the brain activity analysis system 10 according to the first embodiment in processing of a determination unit 23 and a processing unit 24. Therefore, in the configuration according to the third embodiment, points different from the configuration according to the first embodiment will be described.

In a case where a plurality of first ranges is obtained as one estimation result by the first analysis unit 21, the determination unit 23 determines whether a second range overlapping the plurality of first ranges exists. The determination unit 23 according to the first embodiment executes processing in a case where only one first range is estimated, but the determination unit 23 according to the third embodiment executes processing in a case where a plurality of first ranges is obtained as one estimation result by the first analysis unit 21. In this case, the determination unit 23 determines whether there is a second range overlapping any of the plurality of first ranges estimated by the first analysis unit 21.

When there is a second range overlapping any of the plurality of first ranges, the processing unit 24 sets the first range overlapping the second range among the plurality of first ranges as the estimated position. For example, it is assumed that the first analysis unit 21 estimates the first range as a total of three of the first range (1), the first range (2), and the first range (3). In this case, the determination unit 23 determines whether the second range overlaps any of the three first ranges. When the determination unit 23 determines that the second range overlaps one first range (2) among the three first ranges, the processing unit 24 sets the first range (2) as the estimated position. Note that the processing unit 24 may output a position obtained by overlapping the first range (2) and the second range as information regarding the estimated position.

When there is no second range overlapping any of the plurality of first ranges, the processing unit 24 sets the second range as the estimated position. For example, it is assumed that the first analysis unit 21 estimates the first range as a total of three of the first range (1), the first range (2), and the first range (3). However, it is assumed that the determination unit 23 determines that the second range does not overlap any of the three first ranges. In this case, the processing unit 24 outputs the second range as information regarding the estimated position.

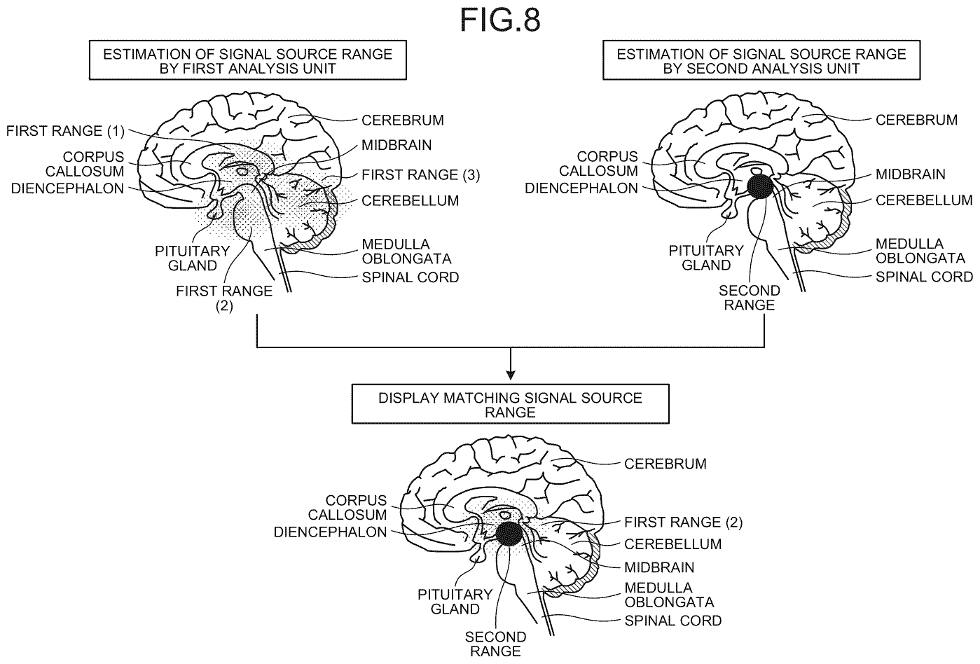

Here, a first display mode of the brain activity analysis system 10 according to the third embodiment will be described with reference to FIG. 8. FIG. 8 is a diagram illustrating the first display mode of the brain activity analysis system according to the third embodiment. As illustrated in FIG. 8, the brain activity analysis system 10 according to the third embodiment determines whether the second range overlaps any of the plurality of first ranges estimated by the first analysis unit 21, and displays the first range overlapping the second range and the second range in an overlapping manner. Although not illustrated in FIG. 8, only the first range overlapping the second range may be displayed. In addition, all the first ranges may be displayed, and the first ranges overlapping the second ranges may be displayed in different modes.

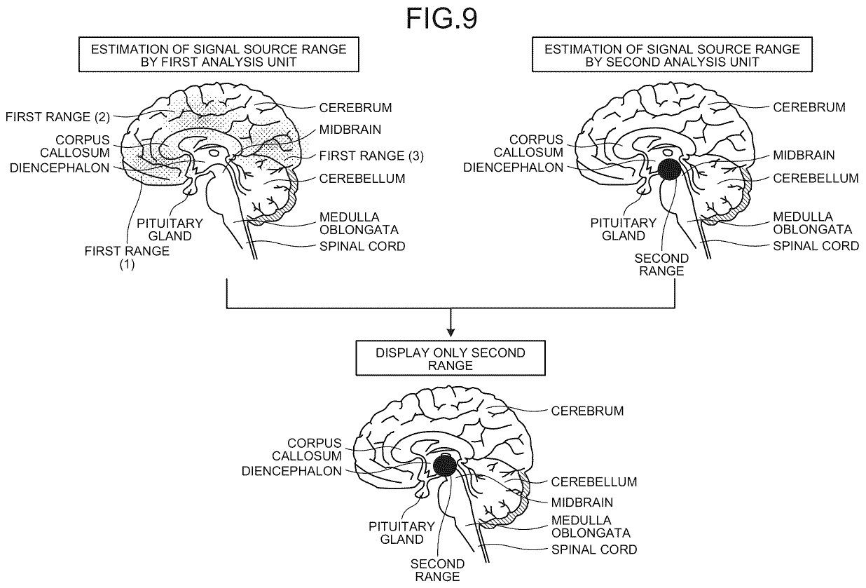

Next, a second display mode of the brain activity analysis system 10 according to the third embodiment will be described with reference to FIG. 9. FIG. 9 is a diagram illustrating the second display mode of the brain activity analysis system according to the third embodiment. As illustrated in FIG. 9, in a case where the second range is not included in any of the plurality of first ranges estimated by the first analysis unit 21, the brain activity analysis system 10 according to the third embodiment displays only the second range.

As a result, even in a case where a plurality of first ranges is estimated by the first analysis unit 21, it is possible to increase the estimation accuracy of the source of the intracerebral signal by outputting the first range in which the second ranges overlap as the information regarding the estimated position.

### Configuration and Effect

The brain activity analysis system 10 according to the present disclosure includes: a first measurement unit 11 configured to measure the intracerebral signal; a second measurement unit 12 configured to measure the biological reaction signal generated in association with the brain activity; a first analysis unit 21 configured to estimate the first range indicating the range of the source of the intracerebral signal based on the measurement data by the first measurement unit 11; a second analysis unit 22 configured to estimate the second range indicating the range of the source of the intracerebral signal based on the measurement data by the second measurement unit 12; and a processing unit 24 configured to calculate the estimated position of the source of the intracerebral signal based on the first range and the second range, and output the information regarding the calculated estimated position.

According to this configuration, the second range indicating the range of the source of the intracerebral signal is estimated based on the biological reaction signal measured by the second measurement unit 12, and both the first range and the second range indicating the source of the intracerebral signal estimated based on the intracerebral signal measured by the first measurement unit 11 are used for estimation of the source of the intracerebral signal, so that the estimation accuracy of the source of the intracerebral signal can be enhanced.

The brain activity analysis system 10 according to the present disclosure further includes a determination unit 23 configured to determine whether the first range and the second range overlap each other, and the processing unit 24 is configured to set the first range as the estimated position when the first range and the second range overlap each other.

According to this configuration, the second range indicating the range of the source of the intracerebral signal is estimated based on the biological reaction signal measured by the second measurement unit 12, and both the first range and the second range indicating the source of the intracerebral signal estimated based on the intracerebral signal measured by the first measurement unit 11 are used for estimation of the source of the intracerebral signal, so that the estimation accuracy of the source of the intracerebral signal can be enhanced.

The brain activity analysis system 10 according to the present disclosure further includes a determination unit 23 configured to determine whether the first range and the second range overlap each other, and the processing unit 24 is configured to set the second range as the estimated position when the first range and the second range overlap each other.

According to this configuration, the second range indicating the range of the source of the intracerebral signal is estimated based on the biological reaction signal measured by the second measurement unit 12, and both the first range and the second range indicating the source of the intracerebral signal estimated based on the intracerebral signal measured by the first measurement unit 11 are used for estimation of the source of the intracerebral signal, so that the estimation accuracy of the source of the intracerebral signal can be enhanced.

The brain activity analysis system 10 according to the present disclosure further includes a determination unit 23 configured to determine whether there is the second range overlapping any of a plurality of first ranges when the first analysis unit 21 obtains the plurality of first ranges as the estimation result, and the processing unit 24 is configured to set the first range overlapping the second range among the plurality of first ranges as the estimated position when there is the second range included in any of the plurality of first ranges.

According to this configuration, even in a case where the plurality of first ranges indicating the source of the intracerebral signal based on the intracerebral signal measured by the first measurement unit 11 are estimated, the second range indicating the range of the source of the intracerebral signal is used for the estimation of the source of the intracerebral signal based on the biological reaction signal measured by the second measurement unit 12, so that the estimation accuracy of the source of the intracerebral signal can be enhanced.

The brain activity analysis method according to the present disclosure includes: measuring the intracerebral signal; estimating the first range indicating the range of the source of the intracerebral signal based on measurement data of the intracerebral signal; measuring the biological reaction signal generated in association with brain activity; estimating the second range indicating the range of the source of the intracerebral signal based on measurement data of the biological reaction signal generated in association with the brain activity; calculating the estimated position of the source of the intracerebral signal based on the first range and the second range; and outputting information regarding the calculated estimated position.

According to this configuration, the second range indicating the range of the source of the intracerebral signal is estimated based on the biological reaction signal measured by the second measurement unit 12, and both the first range and the second range indicating the source of the intracerebral signal estimated based on the intracerebral signal measured by the first measurement unit 11 are used for estimation of the source of the intracerebral signal, so that the estimation accuracy of the source of the intracerebral signal can be enhanced.

A non-transitory computer-readable storage medium stores the program causing a computer to execute: measuring the intracerebral signal; estimating the first range indicating the range of the source of the intracerebral signal based on measurement data of the intracerebral signal; measuring the biological reaction signal generated in association with brain activity; estimating the second range indicating the range of the source of the intracerebral signal based on measurement data of the biological reaction signal generated in association with the brain activity; calculating the estimated position of the source of the intracerebral signal based on the first range and the second range; and outputting information regarding the calculated estimated position.

According to this configuration, the second range indicating the range of the source of the intracerebral signal is estimated based on the biological reaction signal measured by the second measurement unit 12, and both the first range and the second range indicating the source of the intracerebral signal estimated based on the intracerebral signal measured by the first measurement unit 11 are used for estimation of the source of the intracerebral signal, so that the estimation accuracy of the source of the intracerebral signal can be enhanced.

Although the embodiments of the present disclosure have been described above, the embodiments are not limited by the contents of the embodiments. In addition, the above-described constituent elements include those that can be easily assumed by those skilled in the art, those that are substantially the same, and those in a so-called equivalent range. Furthermore, the above-described components can be appropriately combined. Furthermore, various omissions, substitutions, or changes in the constituent elements can be made without departing from the gist of the above-described embodiments.

The present disclosure includes matters that contribute to the realization of “health and wellbeing for all people” of the SDGs (Sutainable Development Goals) and contribute to value creation by healthcare products/services.

The program for implementing the brain activity analysis method according to the present disclosure may be provided by being stored in a non-transitory computer-readable storage medium, or may be provided via a network such as the Internet. Examples of the computer-readable storage medium include optical discs such as a digital versatile disc (DVD) and a compact disc (CD), and other types of storage devices such as a hard disk and a semiconductor memory.

According to the present disclosure, it is possible to provide a brain activity analysis system, a brain activity analysis method, and a program capable of improving estimation accuracy of a source of an intracerebral signal.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A brain activity analysis system comprising:
a first measurement unit configured to measure an intracerebral signal based on a state of a brain;
a second measurement unit configured to measure a biological reaction signal generated in a body site other than the brain in association with brain activity;
a first analysis unit configured to estimate a first range indicating a range of a source of the intracerebral signal based on measurement data by the first measurement unit;
a second analysis unit configured to estimate a second range indicating the range of the source of the intracerebral signal based on the measurement data by the second measurement unit; and
a processing unit configured to calculate an estimated position of the source of the intracerebral signal based on the first range and the second range, and output information regarding the calculated estimated position.

2. The brain activity analysis system according to claim 1, wherein the processing unit is configured to output at least one of the first range and the second range as information regarding the estimated position of the source of the intracerebral signal.

3. The brain activity analysis system according to claim 2, wherein the processing unit is configured to, when the first range and the second range are output, control a display unit to display the first range and the second range in different display modes.

4. The brain activity analysis system according to claim 2, further comprising a determination unit configured to determine whether the first range and the second range overlap each other,
wherein the processing unit is configured to output the first range and the second range as information regarding an estimated position of the source of the intracerebral signal when the first range and the second range do not overlap each other.

5. The brain activity analysis system according to claim 2, further comprising a determination unit configured to determine whether the first range and the second range overlap each other,
wherein the processing unit is configured to output one of the first range and the second range as information regarding an estimated position of the source of the intracerebral signal when the first range and the second range overlap each other.

6. The brain activity analysis system according to claim 5, wherein the processing unit is configured to allow selection of one of the first range and the second range to be output as information regarding an estimated position of the source of the intracerebral signal when the first range and the second range overlap each other.

7. The brain activity analysis system according to claim 5, wherein the processing unit is configured to
output the second range as information regarding an estimated position of the source of the intracerebral signal when the second range is included in the first range, and
output the first range as information regarding an estimated position of the source of the intracerebral signal when the second range is not included in the first range.

8. The brain activity analysis system according to claim 1, further comprising a determination unit configured to determine whether there is the second range overlapping any one of a plurality of first ranges when the first analysis unit obtains the plurality of first ranges as an estimation result,
wherein the processing unit is configured to set the first range overlapping the second range among the plurality of first ranges as the estimated position when there is the second range overlapping any one of the plurality of first ranges.

9. The brain activity analysis system according to claim 1, further comprising a determination unit configured to determine whether there is the second range overlapping any one of a plurality of first ranges when the first analysis unit obtains the plurality of first ranges as an estimation result,
wherein the processing unit is configured to set the second range as the estimated position when there is not the second range overlapping any one of the plurality of first ranges.

10. A brain activity analysis method comprising:
measuring an intracerebral signal based on a state of a brain;
estimating a first range indicating a range of a source of the intracerebral signal based on measurement data of the intracerebral signal;
measuring a biological reaction signal generated in a body site other than the brain in association with brain activity;
estimating a second range indicating the range of the source of the intracerebral signal based on measurement data of the biological reaction signal generated in association with the brain activity;
calculating an estimated position of the source of the intracerebral signal based on the first range and the second range; and
outputting information regarding the calculated estimated position.

11. A non-transitory computer-readable storage medium storing a program causing a computer to execute:
measuring an intracerebral signal based on a state of a brain;
estimating a first range indicating a range of a source of the intracerebral signal based on measurement data of the intracerebral signal;
measuring a biological reaction signal generated in a body site other than the brain in association with brain activity;
estimating a second range indicating the range of the source of the intracerebral signal based on measurement data of the biological reaction signal generated in association with the brain activity;
calculating an estimated position of the source of the intracerebral signal based on the first range and the second range; and
outputting information regarding the calculated estimated position.
